(19) United States
(12) Patent Application Publication
(10) Pub. No.: US 2005/0064018 A1
(43) Pub. Date: Mar. 24, 2005

(54) Use of vitamin D compounds

(76) Inventors: Carlos Simoes-Nunes, Village-Neuf (FR); Gilbert M. Weber, Magden (CH)

Correspondence Address: Stephen M. Haracz, Esq.;BRYAN CAVE LLP, 1290 Avenue of the Americas, New York, NY, 10104, US

(21) Appl. No.: 10/942,172

(22) Filed: Sep. 16, 2004

(30) Foreign Application Priority Data
Sep. 22, 2003 (EP) 03021362.3

(51) Int. Cl. A61K 31/59; A23K 1/165; A23K 1/17; A23K 1/00

(52) U.S. Cl. 424/442; 426/635; 514/167

(57) ABSTRACT

Animal feed with 25-hydroxy vitamin D₃ added, optionally in combination with vitamin D₃, improves bone strength in animals, particularly pigs.

The present invention relates to the use of vitamin D compounds for improving bone strength in animals, particularly pigs. More particularly, the invention relates to the use of 25-hydroxy vitamin D₃, optionally in combination with vitamin D₃, in the manufacture of a food or veterinary composition for improvement of bone strength in animals, particularly pigs. In another aspect, the invention relates to a method of improving bone strength in animals, particularly pigs, which comprises administering to an animal in need of such treatment 25-hydroxy vitamin D₃, optionally in combination with vitamin D₃. In still another aspect, the invention relates to a composition, particularly a pig food or premix therefor, comprising 25-hydroxy vitamin D₃ in combination with vitamin D₃.

In many pig herds locomotor disorders are an important problem. In herds where the value of individual purebred pigs is high or in breeding companies that produce premium hybrid pigs the cost of the mentioned disorders can be high. Osteochondrosis (dyschondroplasia), osteoarthrosis and leg weakness can cause severe crippling, and the frequency of these conditions has been high in pigs with otherwise ideal growth characteristics. These disorders are a worldwide problems that have caused concern in the pig industry for 50 or more years. Thus and accordingly, adequate bone resistance and mineralisation are a permanent objective in swine breeding.

In accordance with the present invention it has been found that the above problems in pig breeding or, more generally, raising of animals can be eliminated or substantially ameliorated by administering to the animals an effective amount of 25-hydroxy vitamin D₃, optionally in combination with vitamin D₃.

25-hydroxy vitamin D₃ and vitamin D₃ (hereinafter: inventive ingredients) are suitably administered together with the food. The term food as used herein comprises both solid and liquid food as well as drinking fluids such as drinking water. Particularly, inventive ingredients can be added as a formulated powder to a premix containing other minerals, vitamins, amino acids and trace elements which is added to regular animal food and thorough mixing to achieve even distribution therein.

In the manufacture of a pig food in accordance with the invention, from about 10 μg/kg to about 100 μg/kg of 25-hydroxy vitamin D₃ and, if required, from about 200 IU/kg to about 4,000 IU/kg of vitamin D₃ are added to regular pig food. Alternatively, a food premix may be prepared on the basis of regular food components by adding active ingredients to such food components in higher concentration, e.g., in a concentration of from about 2 mg/kg to about 20 mg/kg of 25-hydroxy vitamin D₃ and, if required, from about 40,000 IU/kg to about 800,000 IU/kg of D₃. If 5 kg of such premix are added per 1000 kg of regular food this would typically meet the individual need of the animal by normal food consumption. For piglets from 21 to 60 days of age preferable dosages are in the range of from about 10 to 30 μg/kg of 25-hydroxy vitamin D₃ and from about 500 to about 2,000 IU/kg of vitamin D₃. For pigs older than 60 days preferable dosages are in the range of and from about 10 to about 20 μg of 25-hydroxy vitamin D₃ and from about 300 to about 1,200 IU/kg of vitamin D₃.

The invention is further illustrated by the following examples.

### EXAMPLE 1

Growing-fattening pigs of an initial body weight of 64.8±5.03 kg were fed a diet based on maize, barley and soybean meal ad libitum for 34 days. Groups of 16 animals each were fed as follows:
A: basal diet (unmedicated),
B: basal diet+1,200 IU/kg vitamin D₃
C: basal diet+30 μg/kg 25-hydroxy vitamin D₃ (Hy·D® 1.25% Beadlet, Roche Vitamins AG, Basel, Switzerland)
D: basal diet+600 IU/kg vitamin D₃+15 μg/kg 25-hydroxy vitamin D₃ (Hy·D®1.25% Beadlet, Roche Vitamins AG, Basel, Switzerland).

The effects of the addition of 25-hydroxy vitamin D₃ and vitamin D₃ to the diet were determined by measuring the strength (Newton at the breaking point) of the bones of the animals. The results are shown in Table 1.

TABLE 1

| Variables | A (n = 16) | B (n = 16) | C (n = 16) | D (n = 16) |
|---|---|---|---|---|
| Strength - | 423 ± 107^{(1)c} | 463 ± 48^{ce} | 520 ± 72^{df} | 619 ± 147^{df} |
| metacarpals | (100) | (109) | (123) | (146) |
| Strength - | 529 ± 102⁽¹⁾ | 553 ± 169 | 545 ± 105 | 612 ± 92 |
| metatarsals | (100) | (105) | (103) | (116) |
| Strength - | 469 ± 102^{(2)a} | 508 ± 113^{ac} | 533 ± 88^{ac} | 616 ± 119^{bd} |
| metacarpals and | (100) | (107) | (112) | (129) |
| metatarsals | | | | |

⁽¹⁾mean ± standard deviation of the mean of 8 determinations;
⁽²⁾mean ± standard deviation of the mean of 16 determinations;
^{a,b}Mean values within a row with unlike superscript letters were significantly different:
^{a-b}P < 0.01;
^{c-d} and ^{e-f}P < 0.05.

The addition of 25-hydroxy vitamin D₃ and of the combination of 25-hydroxy vitamin D₃ with vitamin D₃ clearly improved the strength of the bones of the growing-finishing pig. The increase of the bone resistant was particularly high with the combination of 25-hydroxy vitamin D₃ with vitamin D₃.

Better bone quality is associated with a reduction of locomotor disorders.

### EXAMPLE 2

Weaner piglets of an initial body weight of 8.4±1.2 kg were fed a diet based on maize, barley and soybean meal ad libitum for 33 days. Groups of 31 animals each were fed as follows:
A: basal diet+1,000 IU/kg vitamin D₃
B: basal diet+2,000 IU/kg vitamin D₃
C: basal diet+40 μg/kg 25-hydroxy vitamin D₃ (Hy·D® 1.25% Beadlet, Roche Vitamins AG, Basel, Switzerland)
D: basal diet+1,000 IU/kg vitamin D₃+20 μg/kg 25-hydroxy vitamin D₃ (Hy·D® 1.25% Beadlet, Roche Vitamins AG, Basel, Switzerland).

The effects of the addition of 25-hydroxy vitamin D₃ and vitamin D₃ to the diet were determined by measuring the strength (Newton at the breaking point) of the bones of the animals. The results are shown in Table 2.

TABLE 2

| Variables | A (n = 31) | B (n = 31) | C (n = 31) | D (n = 31) |
|---|---|---|---|---|
| Strength - femur | 802 ± 126^{(1)a} | 873 ± 123^{ab} | 803 ± 82^{a} | 929 ± 63^{b} |
| | (100) | (109) | (100) | (116) |

⁽¹⁾mean ± standard deviation of the mean of 8 determinations;
^{a,b}mean values within a row with unlike superscript letters were significantly different:
^{a-b}P < 0.05.

In this example the increase of the bone resistant was particularly high with the combination of 25-hydroxy vitamin D₃ with vitamin D₃.

### EXAMPLE 3

A pig food containing 25-hydroxy vitamin D₃ and vitamin D₃ can be prepared as follows:

| Ingredients | [%] |
|---|---|
| Soybean meal | 18 |
| Maize | 53 |
| Barley | 14 |
| Oat meal | 6 |
| Wheat bran | 5.4 |
| Soy oil | 1 |
| Minerals | 1.5 |
| Synthetic amino acids premix | 0.5 |
| Vitamins and trace elements premix | 0.6 |

(containing from about 1.25 g to 5 g of Hy·D® 1.25% Beadlet per 100 g of premix) The ingredients are mixed together and if needed the obtained mash food can be pelleted.

### EXAMPLE 4

A premix for a piglet food containing 25-hydroxy vitamin D₃ and vitamin D₃ can be prepared as follows:

| Ingredients | [%] |
|---|---|
| Hy · D ® 1.25% Beadlet | 0.0320 |
| Vitamin A 500 | 0.8000 |
| Vitamin E 50% | 8.0000 |
| Vitamin D3 500 | 0.0800 |
| Vitamin K3 100% MSB/51% | 0.0800 |
| Vitamin B1 98% | 0.0714 |
| Vitamin B2 80% | 0.1750 |
| Vitamin B6 99% | 0.1212 |
| Vitamin B12 0.1% | 1.0000 |
| Biotin 2% | 0.2000 |
| Folic Acid 80% | 0.0227 |
| Niacin 99.5% | 0.7035 |
| Calpan 98% | 0.4082 |
| Vitamin C | 4.0000 |
| Cholinchloride 60% | 12.0000 |
| Copper sulfate 25% | 12.8000 |
| Iron sulfate 30% | 10.0000 |
| Mangan oxide 62% | 1.6129 |
| Zinc oxide 76% | 5.2632 |
| Cobalt carbonate 5% | 0.0600 |
| Calcium jodate 62% | 0.0323 |
| Sodium selenite 1%/BMP | 0.8001 |
| BHT 100% | 2.0000 |
| Carrier Combination | 6.0000 |
| LACANTES S36400-Z | 10.0000 |
| Limestone | 23.7375 |

All ingredients are carefully mixed together and 0.5% (5 kg/1000 kg of food) of this premix is added to the final piglet food.

Alternatively, 25-hydrox vitamin D3 can also be added in a 1% diluted premix, containing a suitable carrier. Such carrier can be wheat flour, wheat middlings, corn cobbs, rice hulls, almond shells or calcium carbonate alone or in variable mixtures of several of these carriers. A typical formula is:

| Ingredients | [%] |
|---|---|
| Wheat flour | 80.00 |
| Calcium carbonate | 19.68 |
| Hy·D ® 1.25% Beadlet | 0.32 |

All ingredients are carefully mixed together and 0.05% (0.5 kg/1000 kg of food) of this premix is added to the final piglet food.

1. A method of manufacture of a food or veterinary composition for improvement of bone strength in animals comprising admixing 25-hydroxy vitamin D₃ optionally in combination with vitamin D₃, with at least one food or veterinary component to form a food or veterinary composition, respectively.

2. A method as in claim 1 wherein a combination of 25-hydroxy vitamin D₃ and vitamin D₃ is admixed with the at least one food or veterinary component.

3. A method as in claim 1 wherein the food composition is a pig food comprising from about 10 μg/kg to about 100 μg/kg of 25-hydroxy vitamin D₃ and from about 200 IU/kg to about 4,000 IU/kg of vitamin D₃.

4. A method of improving bone strength in pigs which comprises administering to an animal in need of such treatment an amount of about 10 μg/kg to about 30 μg/kg of 25-hydroxy vitamin D₃ and, optionally, about 300 IU to about 2,000 IU of vitamin D₃ per day.

5. A composition comprising 25-hydroxy vitamin D₃ and vitamin D₃.

6. A composition as in claim 5 which is an animal food, particularly for pigs.

7. A composition as in claim 6 comprising from about 10 μg/kg to about 100 μg/kg of 25-hydroxy vitamin D₃ and from about 200 IU/kg to about 4,000 IU/kg of vitamin D₃.

8. A composition as in claim 5 which is a food premix, particularly for pig feed comprising from about 2 mg/kg to about 20 mg/kg of 25-hydroxy vitamin D₃ and from about 40,000 IU/kg to about 800,000 IU/kg of D₃.
